(19) United States
(12) Patent Application Publication
(10) Pub. No.: US 2010/0317736 A1
(43) Pub. Date: Dec. 16, 2010

(54) COMPOSITION OF THE SKIN EXTERNAL APPLICATION OR THE FOOD FOR ACCELERATING PROLINE RECYCLING BY CONTAINING THEANINE

(76) Inventors: Hyun Jung Shin, Seoul (KR); Jeong Kee Kim, Gyeonggi-do (KR); Ji Hae Lee, Gyeonggi-do (KR); Sang Min Lee, Gyeonggi-do (KR); Joo Hyun Baik, Gyeonggi-do (KR)

Correspondence Address: NIXON & VANDERHYE, PC, 901 NORTH GLEBE ROAD, 11TH FLOOR, ARLINGTON, VA, 22203, US

(21) Appl. No.: 12/808,238

(22) PCT Filed: Nov. 28, 2008
(86) PCT No.: PCT/KR2008/007029

§ 371 Date: Aug. 18, 2010

(30) Foreign Application Priority Data
Dec. 17, 2007 (KR) 10-2007-0132054

(51) Int. Cl. A61K 8/44; C07C 229/26; A61Q 19/00; A23F 3/16

(52) U.S. Cl. 514/563; 562/561; 426/597

(57) ABSTRACT

Disclosed herein is a composition for promoting proline recycling, and more particularly, to a composition for external application to skin or cosmetic food comprising theanine as an active ingredient for promoting expression or activity of prolidase, which is an enzyme to promote proline recycling, thereby increasing collagen synthesis and restoring wrinkle.

### TECHNICAL FIELD

The present invention relates to a composition for promoting proline recycling, and more particularly, to a composition for external application to skin or cosmetic food comprising theanine as an active ingredient for promoting expression or activity of prolidase, which is an enzyme to promote proline recycling, thereby increasing collagen synthesis and restoring wrinkle.

### BACKGROUND ART

Green tea is one of drinks world widely popularized and Asia produces the greatest part of the amount consumed in the world. Also, recently, it is one of the most spotlighted natural products because many reports on its usefulness in the body have been reported. It comprises polyphenol, various proteins, amino acids, organic acids, vitamins and mineral pigments. Particularly, as compared to other plants, it comprises polyphenol and amino acids in large amounts and the amounts of such components are known to determine the effect and taste of the tea. Generally, green tea contains substances known for their antioxidant effect and anticancer effect. The most important one of those substances is epigallocatechin gallate (EGCG), a kind of catechin, on which most of researches and studies have been focused.

In addition, theanine (L-theanine) which occupies 1 to 2% of the dried weight of tea leave takes 50% or more of the total amino acids. It is a peculiar component to green tea which is not found in any other plants and is found to be a key component determining the unique taste and effect of green tea. Theanine is biosynthesized by enzymatic reaction of L-glutamine and ethylamine in the root, transferred and concentrated in the leave. Characteristically, it exist in the free fatty acid, though a part of it may be converted into polyphenol by light, and is not utilized as a component of proteins unlike other amino acids. Upon examination of the effects of theanine reported so far, representatively, anticancer activity, effects on the nerve system and stress inhibiting effects are known. For example, it has been reported that it suppresses the excitement caused by intake of caffeine (Toxicol Lett. 2001 123 (2-3), 159-167, Biosci. Biotechnol. Biochem. 2000 February; 64 (2):287-93), increases alpha-waves in the brain to relieve the tension (Nippon Nogeikagaku Kaishi 1998; 72:153-157) and shows anti-stress effects such as suppression of increase of heart rate by stress and reduction of stress response factor (Biol Psychol. 2007 74 (1):39-45). Also, it shows anticancer effect by reducing the glutathione level in the tumor cell (Cancer Lett. 2004 212 (2), 177-184), blood pressure lowering effect (Biosci Biotechnol Biochem 1995 59 (4) 615-618) and anti-oxidation effect of inhibiting oxidation of low density lipidoprotein cholesterol (LDL-cholesterol) (Exp Toxicol Pathol 1997; 49:329-335). Recently, it is studied on its anti-obesitic activity (In Vivo 2004, 18 (1) 55-62) and physiological activity increasing alcohol dehydrogenase (Biol Pharm Bull 2005 28 (9) 1702, 1706). Particularly, it attracts public attention as a material of functional food, cosmetics and medical products, since it is found that it has anti-stress effect and tension relieving effect.

Though concrete results of investigation and verification on the latent physiological effects of theanine have been accumulated, there is no disclosure about effect of theanine on skin, except for the lipolysis effect upon treatment with theanine alone or in combination with green tea-derived catechin (Korean Laid-Open Patent Publication No. 2004-0092538) and the anti-aging effect of theanine (Korean Patent Laid-Open Publication NO. 2007-0028901).

Aging phenomena in the skin such as wrinkles, elasticity loss ad reduction in skin thickness are caused by reduction in the amount of collagen which occupies the large part of the dermal layer in the skin and reduced synthesis of new collagen by deterioration of activity of fibroblasts to synthesize collagen. Collagen is known to vanish by specific external causes such as exposure to UV rays and dryness. As the collagen amount in the dermal layer decreases, the thickness of the dermal layer decreases and finally, wrinkles formed and the skin loses its softness and elasticity. Therefore, in terms of the maintenance of skin health and beauty, it is important to maintain the collagen amount in the skin at a suitable level and to have excellent capability of producing collagen. Various efforts have been made to increase collagen. In order to increase the collagen amount in the dermal layer, it is important to suppress decomposition of collagen existing in the dermal layer. However, it is also important to promote production of new collagen in the fibroblasts. Therefore, the development of a novel substance or composition capable of promoting the collagen synthesis can relieve the aging phenomena such as wrinkles and elasticity loss.

Proline is an amino acid of collagen and is indispensable to the formation of new collagen in the dermal fibroblasts. Collagen has a basic structure of glycine-X-Y as a repeating unit, in which x denotes commonly proline and Y denotes commonly hydroxyproline. Proline in cells is supplied through two routes: one includes conversion of other amino acids such as glutamine by enzymatic reaction and the other is supplied as a by-product of the collagen decomposition. It is disclosed that prolidase, an enzyme, is involved in the last step of the collagen decomposition in the cell and cleaves proline in the C-end of imidodipeptide (The Metabolic Basis of Inherited Disease, New York, McGraw Hill 1989, 577-597) to re-supply proline to the cell. Also, it is reported that by the action of prolidase, the fibroblasts can be rapidly supplied with proline needed for re-synthesis of collagen (Clin. Physiol. Biochem. 7 1989, 128-136) and efficiency of such proline recycling reaches 90% (CMA J. 113 1975, 759-763). Therefore, the second route is predominant over the first route. As to the relation between the collagen synthesis and the prolidase activity, various researches have been conducted (Ann. Academ. Med. Bial. 41 1996, 149-160) and it is known that the prolidase activity is a very important factor in controlling collagen synthesis.

There is no report purposing that theanine derived from green tea can control the prolidase activity to increase the collagen biosynthesis. Also, there is no patent disclosing the activity of theanine to increase the collagen production by prolidase. Though there is a patent disclosing a cosmetic composition of anti-aging effect comprising proline, hydroxyproline and/or a collagen hydrolysis product (France Patent No. 87121832), the patent is only for a simple mixture of collagen components.

### DISCLOSURE

### Technical Problem

Accordingly, the present inventors have conducted researches and studies to seek a compound having the effect to increase prolidase activity of human fibroblasts among natural substances known to not having any particular side effects and found that theanine, a key amino acid of green tea, increases the activity of prolidase which is an important enzyme of the proline recycling to induce proline recycling, thereby promoting proline recycling. Based on the above discovery, the present invention has been completed.

Therefore, it is an object of the present invention to provide a composition for external application to skin or cosmetic food for promoting activity of prolidase in fibroblasts.

### Technical Solution

To achieve the above object, the present invention provides a composition for external application to skin or cosmetic food for promoting proline recycling comprising theanine as an active ingredient.

### ADVANTAGEOUS EFFECTS

The composition for external application to skin or cosmetic food for promoting proline recycling promotes proline recycling and increases collagen biosynthesis in fibroblast by increase of activity of prolidase, thereby showing effects on improvement of skin wrinkle and suppression of photoaging.

### DESCRIPTION OF DRAWINGS

FIG. 1 is a graph showing procollagene production of theanine, vitamin C and transforming growth factor-β (TGF-β).

FIG. 2 is a graph showing cell viability of theanine used according to the present invention.

FIG. 3 is a graph showing prolidase activity according to the concentration of theanine.

FIG. 4 is a graph showing the total amount of synthesized collagen according to the concentration of theanine.

FIG. 5 is a graph showing effects of theanine on promotion of collagen biosynthesis in hairless mouse.

FIG. 6 is a graph showing effects of theanine on wrinkle improvement.

FIG. 7 is photographs showing the skin surfaces of the mice from Example 2 and Comparative Examples 2-3.

FIG. 8 is photographs showing the result of immunohistochemistry using the hairless mice from Example 2 and Comparative Examples 2-3.

### BEST MODE

The present invention is directed to a composition for external application to skin or cosmetic food comprising theanine as an active ingredient which promotes activity of prolidase and increase collagen synthesis.

Now, the present invention is described in detail.

Theanine, as used in the present invention, which is known as an important amino acid of green tea leaf, is a compound represented by the following formula I and is purchased from TAE YANG CHEMICAL, co. in the form purified as a single component.

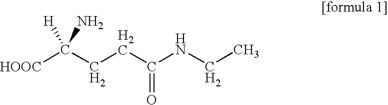

Theanine significantly increases the activity of prolidase when applied to fibroblasts from human. Also, it changes intracellular signaling molecules which are involved in the prolidase activation, and increases collagen synthesis and suppresses photoaging in hairless mice. Therefore, theanine can be usefully used for the improvement of skin wrinkling and the prevention of aging by promoting the activity of prolidase to increase proline recycling and collagen synthesis.

According to the present invention, the composition for promoting proline recycling comprises theanine in an amount of 0.001 to 40 wt %, based on the total weight of the composition. If the amount of theanine is less than 0.001 wt %, it is hard to achieve the expected effects, while if it exceeds 40 wt % there is no significant increase in the effects by the increase in the added amount. Furthermore, according to the present invention, preferably, the composition comprises theanine in an amount of 0.001 to 10 wt % when it is prepared for external application such as creams or lotions and in an amount of 0.1 to 40 wt % when it is prepared for cosmetic food.

According to the present invention, the composition for promoting proline cycling is to promote collagen synthesis.

According to the present invention, the composition for promoting proline cycling is to improve and prevent skin wrinkling.

According to the present invention, the composition for promoting proline cycling is used to improve wrinkling and elasticity of skin and it can be formulated into cosmetic formulations such as skin softener, nourishing toner, massage cream, nourishing cream, pack, gel or adhesive type cosmetics; formulations for transdermal administration such as lotion, ointment, gel, cream, patch or spray; or formulations for oral administration such as pills, capsules, tablets, granules or drinks. The formulation is not particularly limited.

Those skilled in the art, however, can select and combine other ingredients in addition to the essential ingredients according to formulations and intended uses.

Hereinafter, the present invention will be described in further detail with reference to the following examples, but the scope of the present invention is not limited to these examples.

### EXAMPLES

### Experiment Example 1

### Effect of Increasing Procollagen Biosynthesis in Dermal Cells

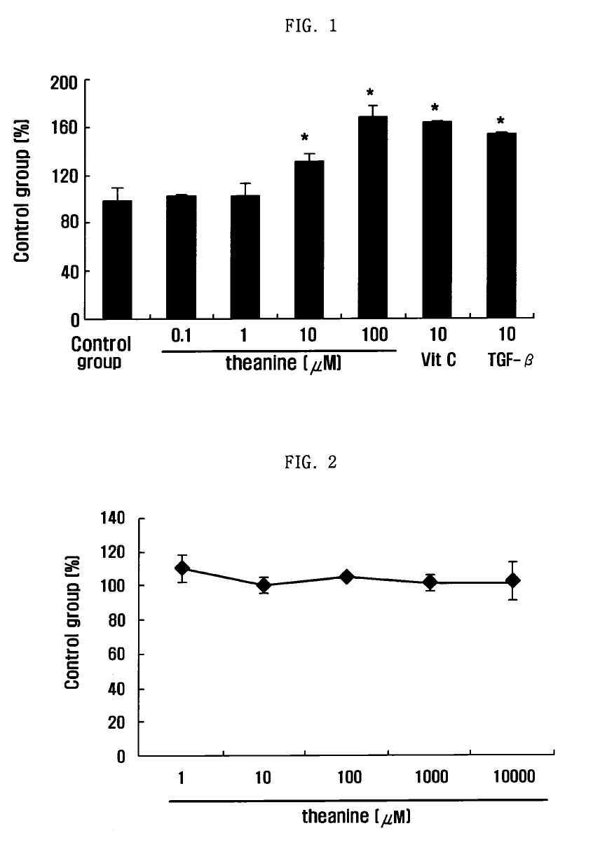

Human fibroblasts are cultured in the concentration of 10⁴ in 48 well plates. The medium of the plates was replaced with a culture medium containing either 10 μM of vitamin C or 10 μM of transforming growth factor-β (TGF-β) and a culture medium containing either 0.1, 1, 10 or 100 μM of theanine. After culturing for 48 hours, the supernatants were harvested and synthesized procollagen was quantified using the ELISA (Takara MK101) method. The data were calculated relative to 100 of control group without containing the above substances and the result was shown in FIG. 1. From the result of FIG. 1, it was noted that theanine increased collagen production of fibroblast concentration-dependently and showed higher procollagen production than the positive control groups containing vitamin C and transforming growth factor-β.

Also, the vitality experiment was performed to examine if the concentration of theanine used in the present invention showed toxicity in vivo. 2×10³ of human fibroblasts were cultured in a 96 well plate, treated with 1, 10, 100, 1000 and 10000 μM of theanine and incubated for 24 hours. 3-(4,5-dimethylazol-2-yl)-2,5-diphenyl-2H-tetraazoliumbromide (MTT) was added to the concentration of 0.5 mg/ml and further incubated for 4 hours. 200 μl of DMSO was treated for the color reaction and the result was shown in FIG. 2.

From the result of FIG. 2, it was confirmed that the concentration of theanine according to the present invention was not toxic at all.

### Experiment Example 2

### Effect of Increasing Prolidase Activity in Dermal Cells

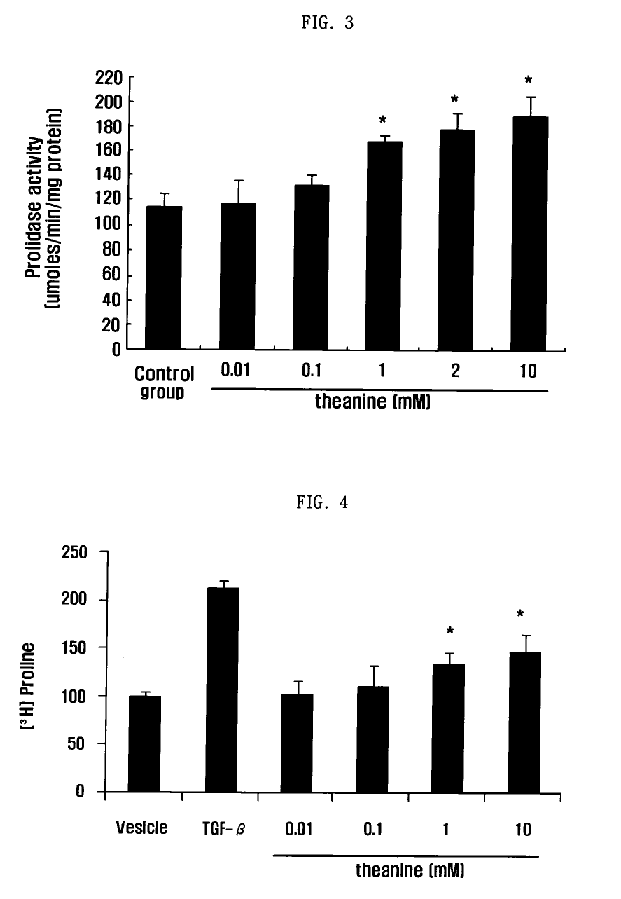

10⁵ of human fibroblasts were cultured in 6 well plates for 6 days. The medium was replaced with heat-inactivated fetal bovine serum and then incubated for an additional 24 hours. The medium was again replaced with a culture medium containing either 0.01, 0.1, 1, 2 or 10 mM of theanine. After 24 hours, the cells were harvested and subjected to the Chinard reaction. The cells were ultrasonically disrupted and centrifuged at 100,000×g for 15 minutes. The supernatant was collected and mixed with 2 mM MnCl₂ in the same amount for the prolidase reaction. The mixture was pre-reacted at 37° C. for 24 hours. 94 mM glycine-proline (Gly-Pro) dipeptide as a substrate of the prolidase reaction was added thereto in the same amount of the pre-reacted mixture and reacted at 37° C. for 1 hours. Then, the reaction was ended by adding 0.45M TCA. The enzymatic activity of prolidase was determined by quantifying proline, which is isolated by prolidase, by the color development using ninhydrin reagent. The enzyme activity was shown as a value calculating the equation of amount of isolated proline over protein weight over time (Mol/min/mg) and the result is shown in FIG. 3.

As shown in FIG. 3, the amount of proline was concentration-dependently increased, that is, the activity of prolidase increased, in the treatment groups, as compared to the control group, which had not been treated with theanine. That is, the activity of prolidase increased. Therefore, it was confirmed that that theanine is effective in promoting recycling proline which is an important amino acid involved in the formation of collagen structure in the cells.

### Experiment Example 3

### Effect of Increasing Total Collagen Biosynthesis in Dermal Cells

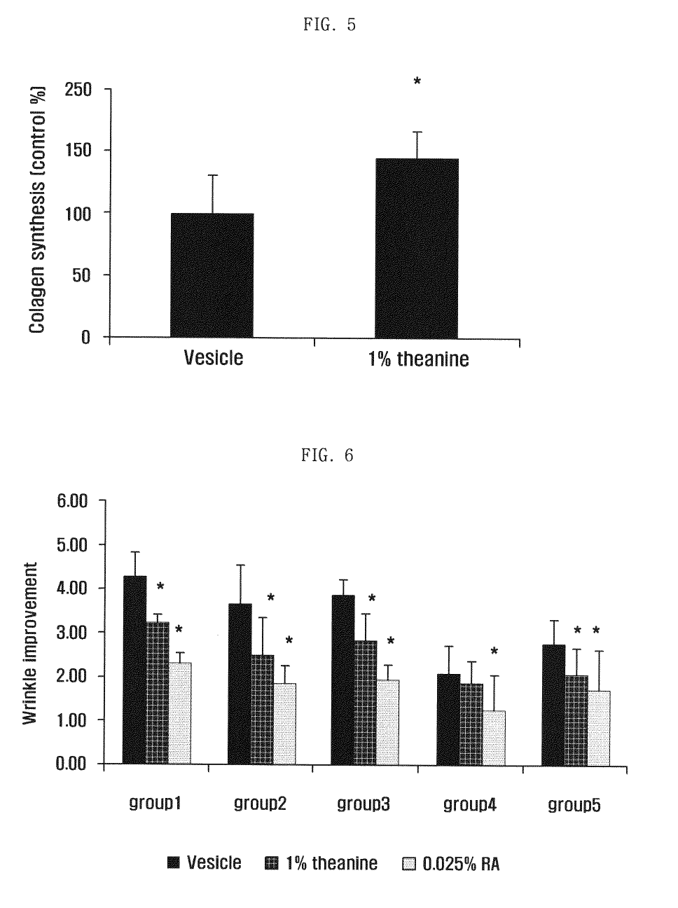

5×10⁴ of human fibroblasts were cultured in 24 well plates. The medium was replaced with a medium containing 164 kBq/ml L-[³H]-proline and supplemented with theanine in a concentration either 0.01, 0.1, 1 or 10 mM. After incubation of 24 hours, the cells were harvested, disrupted and treated with collagenase to separate collagen. Then, the amount of ³H increased by the treatment with theanine was determined using a liquid scintillation counter and compared to that of the control group which had not been treated with theanine. Without regard to the collagen type, the total collagen amount was measured and the data described in FIG. 4 are calculated relative to 100 of the control group. As a result, it was confirmed that the total collagen biosynthesis was increased by about 45% when treated with 1 mM of theanine.

### Example 1 and Comparative Example 1

### Cream

Separately, components of the water phase and the oil phase described in Table 1 were thoroughly dissolved at 70° C. The two phases were mixed together and emulsified at 7,000 rpm for 5 minutes to prepare creams of Example 1 and Comparative Example 1. Unit: weight.

TABLE 1

| | | | Comparative |
|---|---|---|---|
| | Ingredients | Example 1 | Example 1 |
| Oil phase | Beeswax | 2 | 2 |
| | Stearyl alcohol | 5 | 5 |
| | Stearic acid | 8 | 8 |
| | Squalene | 10 | 10 |
| | Propyleneglycol | 3 | 3 |
| | monostearate | | |
| | Polyoxyethylene | 1 | 1 |
| | ether | | |
| | Preservative, | Predetermined | Predetermined |
| | antioxidant | amount | amount |
| Water phase | Glycerin | 4 | 4 |
| | Propyleneglycol | 8 | 8 |
| | Triethylamine | 1 | 1 |
| | Sodium | 0.2 | 0.2 |
| | polyacrylate | | |
| | Purified water | balance | Balance |
| | Theanine | 1 | — |

### Experiment Example 4

### Effect of Increasing Collagen Biosynthesis in Animal Models of Hairless Mice

In order to examine the effect of improving skin wrinkle of theanine, SKH-1 female hairless mice, about 7 weeks old, were subjected to a closed patch test for 2 weeks. Each cream of Example 1 and Comparative Example 1 was applied on the back skin of the hairless mouse and after the closed patch test, the skin was biopsed. Proteins were separated from the biopsed skin and assessed for collagen by western blotting. The result is shown in FIG. 5. From the result of FIG. 5, it was confirmed that theanine increased the collagen biosynthesis in the hairless mice.

### Experiment Example 5

### Effect of Improving Wrinkle in Photoaged Animal Models

In order to examine the effect of improving skin wrinkle of the composition according to the present invention, SKH-1 female hairless mice, about 7 weeks old, were subjected to a topical application test. Each cream of Example 1 and Comparative Example 1 was applied to the back skin of the hairless mice along with UV irradiation. The UV irradiation was performed every two days while measuring MED (Minimal erythema dose) for each animal and the creams of Example 1 and Comparative Example 1 were applied twice a day. The effect of improving wrinkle formation was examined through replica assay. As the positive control group, 0.025% retinoic acid (RA) was used. The replica was divided into 5 parts and analyzed as R1 to R5. The result is shown in FIG. 6. From the result of FIG. 6, it was confirmed that the inhibition of wrinkle formation was improved in the group treated with the cream of Example 1 comprising theanine.

Therefore, it was noted from the results of Experiment Example 1 and Experiment Examples 3 to 5 that theanine used in the present invention increased activity of dermal cells by increasing the activity of prolidase which is important in the collagen biosynthesis, thereby increasing collagen biosynthesis. Also, it was noted that the increase of collagen biosynthesis could inhibit skin wrinkle formation and improve elasticity, and ultimately delay skin aging.

Accordingly, it was noted that the composition for promoting proline recycling comprising theanine according to the present invention could be effectively used as a substance for improvement of the skin aging.

### Experiment Example 6

### Effect of Inhibiting Photoaging in Hairless Mouse

In order to examine the effect of orally administered theanine on photoaging, hairless mice were used as animal models for this experiment. As described in Table 2, 6 to 7 week old female hairless mice (SKH, HR-1) were divided into 3 groups of Comparative Example 2 (normal group), Comparative Example 3 (UV control group) and Example 2 (UV/theanine treatment group), each group having 8 mice and were raised for the experiment period.

TABLE 2

| | Administration |
|---|---|
| Comparative Example 2 | Normal group, 0.5 ml of saline |
| Comparative Example 3 | UV control group, 0.5 ml of |
| | saline |
| Example 2 | UV irradiation, theanine |
| | dissolved in 0.5 ml of saline |

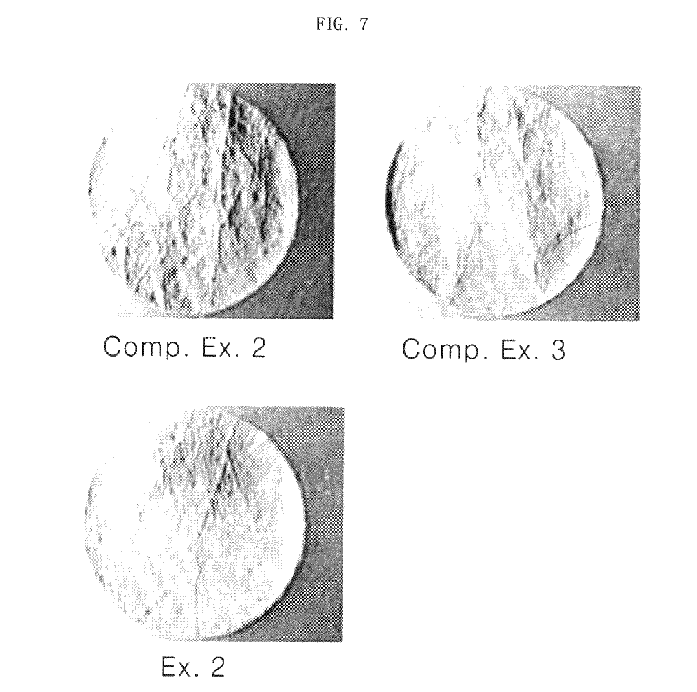

The mice of Comparative Example 2 and Comparative Example 3 were orally administered with 0.5 ml of saline and the mice of Example 2 were administered with 500 mg of theanine, based on the solid faction, per kg of body weight dissolved in 0.5 ml of saline using a syringe for liquid injection. The administration was continued for total 5 weeks and performed at the predetermined time for 5 days per week. From 2 weeks through 5 weeks after the administration had started, the groups of Comparative Example 3 and Example 2 were irradiated by UV under conditions similar to solar light three times per week. Here, the total UV irradiation for the experiment period was set to 600 mJ/cm². In order to objectively judge the effect of improving wrinkle, a replica was taken from the back of each hairless mouse prior to an autopsy. The skin surface was imaged using SkinVisiometer to compare and rates the degree of the skin wrinkles. The result is shown in FIG. 7. From the result of FIG. 7, the density and depth of the wrinkles on the skin surface of the hairless mice of Example 2 were alleviated as compared to those of Comparative Example 3.

### Experiment Example 7

### Estimation of Effect of Inhibiting Photoaging by Tissue Staining

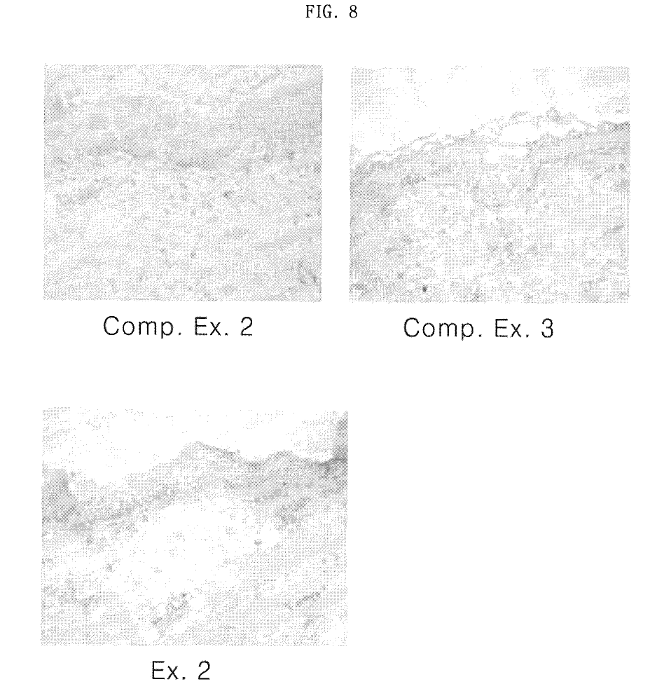

This example was performed using the hairless mice of Example 2 and Comparative Example 2 to 3, which had been used in Experiment Example 6 and histopathological observation was performed by immunohistochemistry. The skin on the back of the hairless mice was taken, fixed in 10% neutral formalin and subjected to immunohistochemistry using monoclonal IgG1 antibody to examine the expression of type 1 collagen in the skin tissue. The result is shown in FIG. 8.

FIG. 8 shows the result of the immunohistochemistry of type 1 collagen in the skin tissue of each experiment group. It was noted that less collagen was stained in Comparative Example 3 (UV control group), as compared to Comparative Example 2 (normal group), while more collagen was stained in the epidermal and dermal junction in Example 2 (UV/theanine treatment group), as compared to Comparative Example 3. Therefore, it was confirmed that the administration of theanine could increase collagen synthesis in the skin.

In addition, the H&E (heamatoxylin & eosin) staining assay was performed for observation of general tissue condition and measurement of thickness of the epidermal layer. The thickness of the epidermal layer was measured at 10 different spots randomly selected per tissue by observing the H&E stained slide under the microscope with 100× magnification and the average value was shown in Table 3.

TABLE 3

| | Thickness of dermal layer |
|---|---|
| Comparative Example 2 | 5.23 ± 0.31 mm (53.5%) |
| Comparative Example 3 | 9.77 ± 0.68 mm (100%) |
| Example 2 | 7.56 ± 0.75 (77.4%) |

From the result of Table 3, it was confirmed that the UV/theanine treatment group (Example 2) showed decrease in the thickness of the epidermal layer by about 23%, as compared to the UV control group (Comparative Example 3) and thus, the administration of theanine could alleviate the skin thickening by UV irradiation.

### Example 3

### Pills

The components described in Table 4 were mixed to prepare pills using a pill making machine. The final weight of the contents was 4 g/pill. Unit:wt %.

TABLE 4

| Theanine | lactose | Glycerin | xylitol |
|---|---|---|---|
| 1 | 1.5 | 1 | 0.5 |

### Comparative Example 4

Pills were prepared by following the procedure described in Example 3, except for using glucose instead of theanine in the same amount.

### Experiment Example 8

### Simple Clinical Examination

40 adult women of 25 to 45 years old were divided into 2 groups, in which one was administered with 30 pills, prepared in Example 3, per day for 30 days and the other, the control group, was administered with the pills prepared in Comparative Example 4 by the same method. After completion of the test, the participants were asked to answer a questionnaire and the result is shown in Table 5.

TABLE 5

| | Example 3 | | | | Comparative Example 4 | | | |
|---|---|---|---|---|---|---|---|---|
| | 20s | 30s | 40s | Total | 20s | 30s | 40s | Total |
| Questionnaire | (4) | (10) | (6) | (20) | (6) | (8) | (6) | (20) |
| Moist feeling | 3 | 8 | 5 | 16 | 2 | 1 | 2 | 5 |
| | | | | (80%) | | | | (25%) |
| Elasticity | 2 | 8 | 5 | 15 | 1 | 2 | 1 | 4 |
| | | | | (75%) | | | | (20%) |
| Reduced | 2 | 8 | 4 | 14 | 1 | 3 | 2 | 6 |
| wrinkle | | | | (70%) | | | | (30%) |
| Make-up | 3 | 9 | 5 | 17 | 2 | 2 | 3 | 7 |
| application | | | | (85%) | | | | (35%) |
| Improvement | 3 | 7 | 4 | 14 | 2 | 3 | 1 | 6 |
| of general | | | | (70%) | | | | (30%) |
| skin | | | | | | | | |
| condition | | | | | | | | |

From the result of Table 5, it was shown that Example 3 is higher in dampness and elasticity of skin, as compared to Comparative Example 4. Also, more experiment participants felt fine wrinkles reduced and responded that the make-up is well applied and the general skin condition was improved. Therefore, it was concluded that the skin improvement effect of theanine was significant.

The cosmetic composition for oral administration comprising theanine can be formulated into various formulation types including the following Examples, but not limited thereto.

### Example 4

### Soft Capsules

100 mg of theanine, 50 mg of soybean extract, 180 mg of soybean milk, 50 mg of red ginseng extract, 2 mg of palm oil, 8 mg of hydrogenated palm oil, 4 mg of yellow beeswax and 6 mg of lecithin were mixed. A single soft capsule was filled with 400 mg of the mixture by following the common method.

### Example 5

### Tablets

100 mg of theanine, 50 mg of soybean extract, 100 mg of glucose, 50 mg of red ginseng extract, 96 mg of starch and 4 mg of magnesium stearate were mixed and 40 mg of 30% ethanol was added thereto. The mixture is formed into granules, dried at 60° C. and shaped into tables using a tablet press.

### Example 6

### Granulates

100 mg of theanine, 50 mg of soybean extract, 100 mg of glucose, 50 mg of red ginseng extract and 600 mg of starch were mixed and 100 mg of 30% ethanol was added thereto. The mixture was formed into granules, dried at 60° C. and put into a bag. The final weight of the content was 1 g.

### Example 7

### Dinks

100 mg of theanine, 50 mg of soybean extract, 10 g of glucose, 50 mg of red ginseng extract, 2 g of citric acid and 187.8 g of purified water were mixed and a bottle was filled with the mixture. The final volume of the contents was set to 200 ml.

1. A composition for external application to skin to accelerate proline recycling comprising theanine as an active ingredient.

2. A composition for a cosmetic or food to accelerate proline recycling comprising theanine as an active ingredient.

3. The composition for external application to skin of claim 1, wherein the theanine is contained in an amount of 0.001 to 10 wt %, based on the total weight of the composition.

4. The composition for a cosmetic or food of claim 2, wherein the theanine is contained in an amount of 0.1 to 40 wt %, based on the total weight of the composition.

5.-9. (canceled)

10. A method of accelerating collagen synthesis.

11. A method of combating skin wrinkles, loss of skin elasticity or reduction of skin thickness comprising topically applying to the skin a composition comprising theanine as an active ingredient.

12. A method for accelerating proline recycling of the skin comprising topically applying to the skin a composition comprising theanine as an active ingredient.

13. A method of accelerating collagen synthesis comprising topically applying to the skin a composition comprising theanine as an active ingredient.
